(19) United States
(12) Patent Application Publication
(10) Pub. No.: US 2003/0126650 A1
(43) Pub. Date: Jul. 3, 2003

(54) MUTANT APICAL DOMINANCE GENE IN EUSTOMA

(76) Inventors: CLAUDE HOPE, CARTAGO (CR); ELLEN F. LEUE, DEKALB, IL (US); LYNNE KNOSHER, SAINT CHARLES, IL (US)

Correspondence Address: ROCKEY MILNAMOW & KATZ LTD, 500 W. MADISON STREET, SUITE3800, CHICAGO, IL, 60661-4592, US

(21) Appl. No.: 09/429,723

(22) Filed: Oct. 29, 1999

Related U.S. Application Data
(60) Provisional application No. 60/106,510, filed on Oct. 30, 1998.

(51) Int. Cl. A01H 5/00

(52) U.S. Cl. 800/323

(57) ABSTRACT

The present invention relates to new, distinct and stable Eustoma cultivars of which exhibit reduced apical dominance. The Eustoma cultivars of the present invention contain an allele which produces a plant having a reduced apical dominance.

### RELATED APPLICATION INFORMATION

This application is a continuation-in-part of U.S. application No. 60/106,510, filed on Oct. 30, 1998.

### FIELD OF THE INVENTION

The present invention relates to an apical dominance allele. In Eustoma, this allele results in plants which exhibit a reduction in apical dominance and hence, an increase in basal and total branching. The present invention also relates to Eustoma seed, a Eustoma plant, a Eustoma variety and a Eustoma hybrid which contain this allele. In addition, the present invention also relates to methods for transferring this allele in a Eustoma plant to other Eustoma varieties and species and is useful for producing novel types and varieties of Eustoma which exhibit a reduction in apical dominance and hence, an increase in basal and total branching.

### BACKGROUND OF THE INVENTION

Eustoma (Eustoma grandiforum), commonly known as prairie gentian or Texas Bluebell, originated as a common North American wildflower. Originally botanists categorized Eustoma grandiflorum in the Lisianthus genus. Although the classification was changed, it is still referred to commonly as lisianthus. Eustoma is used as an annual herbaceous ornamental crop introduced into cultivation as a flowering-pot plant, bedding plant and a cut flower. It is available in a range of colors including white, light yellow, pink, rose, lilac and deep purple-blue to a variety of bicolors. Flowers can be single or double.

At present, high light areas are needed to produce a well-branched pot Eustoma. It would be desirable to produce a Eustoma having increased branching to broaden production into lower light areas. In addition, increased branching in a Eustoma would offer better show in the garden through improved habit and associated increased flower production. Such a trait incorporated into cut flower Eustoma could increase cut stem yield per plant.

### SUMMARY OF THE INVENTION

The present invention relates to a Eustoma plant having reduced apical dominance. The Eustoma plant of the present invention contains an allele for reduced apical dominance. Additionally, the Eustoma plant of the present invention has a pedigree which includes the plant 752 or derivatives thereof, such as, but not limited to, 1389-1-1-9-4-3, 3000-1-2-1-1-1, 3107-1-1-3, 3130-1-3-1-1, 3030-1-4-2-5 or 3000-2-3-5-ml-2-2. The present invention also relates to seed, pollen, ovules, tissue culture and cuttings of a Eustoma plant having reduced apical dominance.

The present invention further relates to Eustoma seed containing an allele for reduced apical dominance. The Eustoma seed of the present invention also has a pedigree which includes the plant 752 or derivatives thereof, such as, but not limited to, 1389-1-1-9-4-3, 3000-1-2-1-1-1, 3107-1-1-3, 3130-1-3-1-1, 3030-1-4-2-5 or 3000-2-3-5-ml-2-2. The present invention also relates to a plant produced by growing the above-described seed.

The present invention also relates to a reduced apical dominance allele which when expressed in a Eustoma plant produces a reduced apical dominant phenotype in said plant.

The present invention further relates to a method for producing F₁ hybrid Eustoma seed. The method involves crossing a first parent Eustoma plant with a second parent Eustoma plant and harvesting the resultant F₁ hybrid Eustoma seed. The first and second parent Eustoma plants used in this method exhibit reduced apical dominance. The present invention also relates to a first generation (F₁) hybrid plant which is produced by growing hybrid Eustoma seed produced by the above-described method.

The present invention also relates to a process for identifying a Eustoma plant, the genome of which contains an allele for reduced apical dominance. This process involves crossing a Eustoma plant to be tested with a Eustoma plant, the genome of which contains an allele for reduced apical dominance, recovering the resulting F₁ hybrid Eustoma seed, planting the F₁ hybrid Eustoma seed and regenerating into plants and selecting Eustoma plants, the genome of which contains the allele for reduced apical dominance, and which exhibit a reduced apical dominance phenotype.

The present invention also relates to a process of obtaining hybrid Eustoma seed, the genome of which contains an allele for reduced apical dominance. The process involves crossing a Eustoma plant obtained using the hereinbefore described process with a Eustoma plant, the genome of which contains an allele for reduced apical dominance and recovering the resulting hybrid Eustoma seed, the genome of which contains an allele for reduced apical dominance.

The present invention also relates to a process for obtaining a hybrid Eustoma plant the genome of which contains an allele for reduced apical dominance. The process involves crossing a Eustoma plant obtained using the hereinbefore described process with a Eustoma plant, the genome of which contains an allele for reduced apical dominance, recovering the resulting hybrid Eustoma seed, the genome of which contains an allele for reduced apical dominance and planting the hybrid Eustoma seed and regenerating into plants, the genome of which contains the allele for reduced apical dominance.

Finally, the present invention relates to viable Eustoma seeds and plants and succeeding generations thereof grown from seeds deposited under ATCC Accession number 203392 and Eustoma seeds and plants to which the allele for reduced apical dominance is transferred from the deposited seeds in succeeding generations thereof

### BRIEF DESCRIPTION OF THE FIGURES

FIG. 1A shows the Eustoma cultivars of the present invention which contain the reduced apical dominance allele and which exhibit an increase in basal and total branching.

FIG. 1B show Eustoma plants which do not contain the reduced apical dominance allele. These plants do not exhibit an increase in basal and total branching.

FIG. 2 shows the RAD Eustoma hybrid 3087 (labeled as “A” in the photograph) compared to commercial hybrids “Blue Lisa” (labeled as “B” in the photograph) and “Florida Blue” (labeled as “C” in the photograph).

FIG. 3 shows the RAD Eustoma hybrids 3214 (labeled as “A” in the photograph), 3213 (labeled as “B” in the photograph) and 3211 (labeled as “C” in the photograph) compared to the commercial hybrid “White Lisa” (labeled as “D” in the photograph).

FIG. 4 shows the RAD Eiustoma hybrid 3215 (labeled as “A” in the photograph) compared to the commercial hybrids “Pink Lisa” (labeled as “B” in the photograph) and “Florida Pink” (labeled as “C” in the photograph).

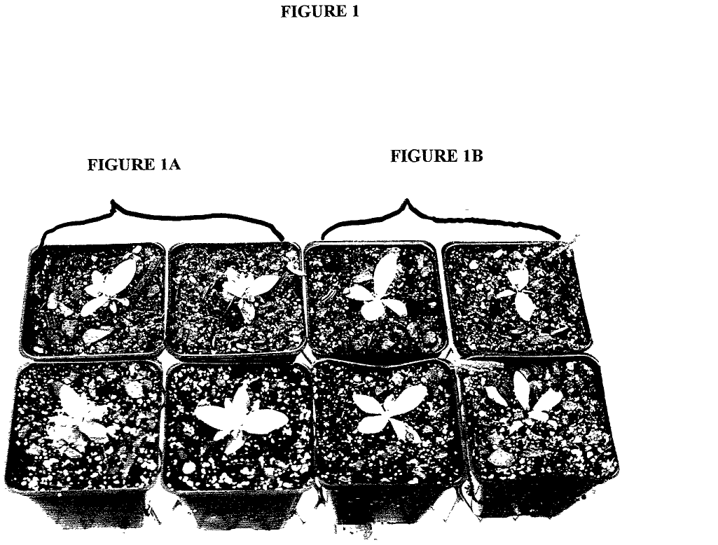

### DETAILED DESCRIPTION OF THE INVENTION

The Eustoma cultivars of the present invention exhibit a substantial reduction in apical dominance. A transferrable allele which results in reduced apical dominance and hence, increased basal and total branching, has been discovered and incorporated into other genetic backgrounds. As used herein, the term “reduced apical dominance” (hereinafter referred to as “RAD”), means that apical dominance is reduced such that plants expressing this trait have increased basal branching when compared with commercial hybrids. As used herein, the term “basal branching” refers to the branches arising from the cotyledonary node below the first true leaves. The RAD hybrids also demonstrate high levels of total branching. As also used herein, the term “total branching” refers to shoots arising from the basal branches and from the main stem above the first true leaves. Additionally, multiple branches per leaf axil are present under high light conditions. As used herein, the term “high light conditions” means that the amount of light present per 24 hour day is from about 20 to about 30 mole·m⁻²·day⁻¹.

Eustoma cultivars which do not contain the allele described herein branch poorly when grown under low light conditions. As used herein, the term “low light conditions” means that the amount of light present per 24 hour day is from about 5 to about 6 mole·m²·day⁻¹. In contrast, Eustoma cultivars containing the RAD allele described herein branch under low light conditions.

The RAD allele was discovered as a result of breeding and research efforts which were conducted at Linda Vista, Costa Rica. In 1987, a cross was made using a Eustoma line received from the United States Department of Agriculture. This Eustoma line did not exhibit any reduced apical dominance and was disease susceptible. This line was crossed with a second, proprietary Eustoma line which did not exhibit any reduced apical dominance. Seed from this cross was collected and planted. In the resulting F₁ population, none of the resulting plants exhibited any reduced apical dominance. The plants of the F₁ population were then self-fertilized. The seed from this self-fertilization was collected and planted. In the resulting segregating F₂ population, a single mutant inbred Eustoma line, designated 752, was discovered which exhibited reduced apical dominance. The remaining plants in this segregating population did not exhibit any reduced apical dominance. Proprietary inbred Eustoma line 752 exhibits the RAD trait, is only about 12 cm in height and is susceptible to many types of diseases. A breeding program was begun using proprietary inbred Eustoma line 752 to introduce the RAD trait into other Eustoma genetic backgrounds. Specifically, this proprietary inbred line was crossed with other Eustoma lines containing commercially desirable traits, such as improved disease resistance. It may also be transferred to taller Eustoma plants to increase the cut stem yield, or to create vigorous branched garden plants. Additionally, polyploid Eustoma plants can be created through various methods, including application of colchicine, protoplast fusion, and other well known techniques.

Based on their breeding and research efforts as described herein, and while not wishing to be bound by any theory, the inventors believe that the RAD trait described herein is controlled by a single recessive gene. However, it is contemplated that it is possible that in certain genetic backgrounds that the RAD trait may have a more complex inheritance.

The Eustoma cultivars of the present invention are genetically stable and can be stably reproduced by means of asexual or sexual propagation. Additionally, the RAD allele which conveys this reduced apical dominance can and has been, bred into diverse Eustoma genetic backgrounds.

The Eustoma cultivars of the present invention maintain functional male and female organs, thus making the incorporation/introgression of the RAD trait into the genetic backgrounds of other Eustoma cultivars possible. Introgression of RAD gene into a desired genetic background can be performed by applying pollen from one plant to the exposed stigma of the flower of another plant. The trait for RAD may be incorporated into cultivars with many different flower colors, including cultivars with bicolor flowers, as well as into cultivars having single, double and semi-double flowers. Well-known Eustoma plants having such characteristics are readily available.

After obtaining Eustoma plants having a desired blend of traits, these plants can be propagated using standard methods known in the art. Specifically, it is expected that any selected RAD Eustoma can be produced commercially through asexual propagation. All RAD Eustoma tested thus far have been found to be stable through asexual propagation. Cuttings for asexual propagation can be taken at any time of the year and no special soil mixtures are required. Hormones, such as, indole-3-butyric acid (referred to as “IBA”) or naphthaleneacetic acid (referred to as “NAA”) may be used as needed.

Additionally, it has been demonstrated that the Eustoma cultivars of the present invention can be produced as progeny from sexual crosses for the purposes of selling seed. Methods for the storage of such seed are well known in the art.

Since, as described hereinbefore, the RAD trait appears to be controlled by a single recessive gene, it is possible to determine whether a particular plant carries the RAD gene by performing an allelism test. This test is performed by crossing the test plant with a plant exhibiting the RAD trait. If the product of the cross also has the RAD trait, then the test plant carries the RAD gene. On the other hand, if none of the hybrid progeny exhibit a substantial reduction in apical dominance, then the branching of the test plant is not due to the RAD gene.

By way of example, and not of limitation, examples of the present invention shall now be given.

### EXAMPLE 1

### Reduced Apical Dominance in Blue Hybrid Eustoma 3087

Eustoma hybrid 3087 is a cross between two reduced apical dominant lines, 1389-1-1-9-4-3 (hereinafter, the “1389 line”)×3000-1-2-1-1-1 (hereinafter, the “3000” line). The 1389 line has a height of 30 cm and was derived from a cross between Eustoma line, 5-2-5-4, and Eustoma line 752. Line 5-2-5-4 has flowers which are ivory in color, a height of about 45 cm and is disease resistant. Seed from this cross was collected and planted. An individual F₁ plant was selected and self-pollinated, and the resulting seed was collected and planted. In the resulting F₂ population, a plant containing the reduced apical dominant character was selected. This selected plant, referred to as 1389-1-1, was then self-fertilized for three (3) generations.

The 3000 line was derived from a cross between Eustoma line 752 and “White Lisa” (developed by and commercially available from Pan American Seed Company, 622 Town Road, West Chicago, Ill. 60185). “White Lisa” is about 16 cm in height and was derived from a cross between 31-14-1-2-9 which has ivory flowers and is about 16 cm in height and 13-25-2-1-9 which has ivory flowers and is about 16 cm in height. Seed from this cross was collected and planted. An individual F₁ plant was selected and self-pollinated, and the resulting seed was collected and planted. In the resulting F₂ population, a plant containing the reduced apical dominant character was selected. This selected plant, referred to as 3000-1-2, was then self-fertilized for three (3) generations.

Eustoma hybrid 3087 contains the RAD allele and is a multiple branched semi-dwarf Eustoma having a height of about 30 cm. The branching of this hybrid starts when the hybrid is in the plug stage. The flower is a campanulate type having a diameter of about 5.5 cm and is deep blue in color (2617C in the Pantone Color Selector 1000 Coated 5^{th} Printing 1993-1994) with shading to lighter blue in the center. The leaves of this Eustoma are 4×3 cm (length×width) and are medium green (POLT-C in the Pantone Color Selector 1000 Coated 5^{th} Printing 1993-1994) in color. Additionally, Eustoma hybrid 3087 exhibits some resistance to soil borne diseases.

Eustoma hybrid 3087 can be used in sexual crossings to introgress the RAD gene into the genetic backgrounds of other Eustoma plants having commercially desirable characteristics in order to create new plants exhibiting the RAD trait.

### EXAMPLE 2

### Reduced Apical Dominance in Light Sky Blue Eustoma Hybrid 3212

Eustoma hybrid 3212 is a cross between two reduced apical dominant lines, 3107-1-1-3 (hereinafter, the “3107 line”)×3130-1-3-1-1 (hereinafter, the “3130 line”). The 3107 line has a height of 16 cm and was derived from a cross between Eustoma line, 3003-1-1-1-3 and Eustoma line 3026-2-3. 3003-1-1-1-3 has flowers which are pink in color, a height of about 16 cm and was derived from a cross between 752×JP29. Seed from this cross was collected and planted. An individual F₁ plant was selected, and self-pollinated and the resulting seed was collected and planted. In the resulting F₂ population, a plant containing the reduced apical dominant character was selected. This selected plant, referred to as 3107-1-1, was then self-fertilized for one (1) generation.

The 3130 line was derived from a cross between Eustoma line 3179-1-2-4 and 3026-3-2. 3026 is about 16 cm in height and was derived from a cross between Eustoma line 3013-1 which has sky blue flowers and is about 19 cm in height and Eustoma line 3000-2-3-2 which has blue flowers and is about 16 cm in height. The 3000 line was derived from a cross between 752×“White Lisa”. Seed from this cross was collected and planted. An individual F₁ plant was selected and self-pollinated, and the resulting seed was collected and planted. In the resulting F₂ population, a plant containing the reduced apical dominant character was selected. This selected plant, referred to as Eustoma line 3130-1-3, was then self-fertilized for two (2) generations.

Eustoma hybrid 3212 contains the RAD allele and is a multiple branched dwarf Eustoma having a height of about 16 cm. The branching of this hybrid starts when the hybrid is in the plug stage. The flower is a campanulate type having a diameter of about 5.25 cm and is light sky blue in color (ECOO-C, 2706C Pantone Process Color Imaging Guide 1000, 1992).

The leaves of this Eustoma are 6×3.5 cm (length×width) and are medium green.

Eustoma hybrid 3212 can be used in sexual crossings to introgress the RAD gene into the genetic backgrounds of other Eustoma plants having commercially desirable characteristics in order to create new plants exhibiting the RAD trait.

### EXAMPLE 3

### Reduced Apical Dominance in Ivory Eustoma Hybrid 3213

Eustoma hybrid 3213 is a cross between two reduced apical dominant lines, 3030-1-4-2-5 (hereinafter, the “3030 line”)×3000-2-3-5-ml-2-2 (hereinafter, the “3000” line). The 3030 line has a height of 19 cm and was derived from a cross between Eustoma line 3000-3-1 and Eustoma line 13539-3. 3000-3-1 (which is a cross between 752×“White Lisa”) has flowers which are ivory in color, a height of about 16 cm and is disease resistant. Seed from this cross was collected and planted. An individual F₁ plant was selected and self-pollinated, and the resulting seed was collected and planted. In the resulting F₂ population, a plant containing the reduced apical dominant character was selected. This selected plant, referred to as 3030-1-4, was then self-fertilized for two (2) generations.

The 3000 line was derived from a cross between Eustoma line 752 and “White Lisa”. Seed from this cross was collected and planted. An individual F₁ plant was selected and self-pollinated, and the resulting seed was collected and planted. In the resulting F₂ population, a plant containing the reduced apical dominant character was selected. This selected plant, referred to as 3000-2-3, was then self-fertilized for one (1) generation. Then the seed was massed and selfed for two (2) subsequent generations.

Eustoma hybrid 3213 contains the RAD allele and is a multiple branched dwarf Eustoma having a height of about 16 cm. The branching of this hybrid starts when the hybrid is in the plug stage. The flower is a campanulate type having a diameter of about 5.5 cm and is ivory in color (lighter than OOCO-C, 607C in the Pantone Process Color Imaging Guide 1000, 1992).

The leaves of this Eustoma are 5×3.5 cm (length×width) and are medium green.

Eustoma hybrid 3213 can be used in sexual crossings to introgress the RAD gene into the genetic backgrounds of other Eustoma plants having commercially desirable characteristics in order to create new plants exhibiting the RAD trait.

### EXAMPLE 4

### Comparison of Reduced Apical Dominance Hybrids to Commercial Hybrids

Five greenhouse plants of each hybrid listed in Table 1, below, were evaluated for basal branching and total branching. Plants were grown using standard greenhouse procedures and exposed to approximately 20 mole·m²·day⁻¹ each day. The plants were evaluated after approximately three and one-half months growth for basal branching and after approximately four and one-half months growth for total branching. Commercial hybrids include the “Lisa” series and the “Florida” series both marketed by Pan American Seed, 622 Town Road, West Chicago, Ill. 60185. Basal branches are the branches arising from the cotyledonary node below the first true leaves. Total branches include shoots arising from the basal branches and from the main stem above the first true leaves. Only branches longer than 2.5 cm were included in this count. Means were compared using the Least Significant Difference Test (α=0.05). Statistical analysis demonstrates that all hybrids having the Reduced Apical Dominance trait have significantly higher basal branching and total branching than the commercially available hybrids.

TABLE 1

| | Basal | LSD = 1.02 | Total | LSD = 2.62 |
|---|---|---|---|---|
| Hybrid | Branches | α = .05 | Branches | α = .05 |
| “Blue Lisa” | 0 +/− 0 | a | 1.0 +/− 0 | a |
| “White Lisa” | 0 +/− 0 | a | 2.2 +/− 0.45 | ab |
| “New Pink Lisa” | 0 +/− 0 | a | 1.8 +/− 0.84 | ab |
| “Florida Blue” | 0 +/− 0 | a | 1.6 +/− 0.89 | ab |
| “Florida Sky Blue” | 0.2 +/− 0.45 | a | 2.6 +/− 0.55 | b |
| “Florida Pink” | 0.2 +/− 0.45 | a | 1.8 +/− 0.84 | ab |
| 3087 | 4.4 +/− 0.55 | bcd | 9.6 +/− 1.14 | gh |
| 3091 | 3.6 +/− 1.14 | bc | 5.2 +/− 0.84 | c |
| 3094 | 4.4 +/− 0.89 | bcd | 10.2 +/− 2.17 | h |
| 3210 | 3.8 +/− 0.45 | bcd | 6.8 +/− 1.3 | de |
| 3212 | 4.8 +/− 0.45 | d | 8.8 +/− 2.17 | gh |
| 3213 | 4.6 +/− 1.14 | cd | 9.0 +/− 1.22 | gh |
| 3214 | 4.8 +/− 1.1 | d | 7.2 +/− 1.1 | ef |
| 3211 | 3.4 +/− 1.67 | b | 8.4 +/− 1.52 | fg |
| 3215 | 4.2 +/− 1.3 | bed | 5.4 +/− 1.14 | cd |

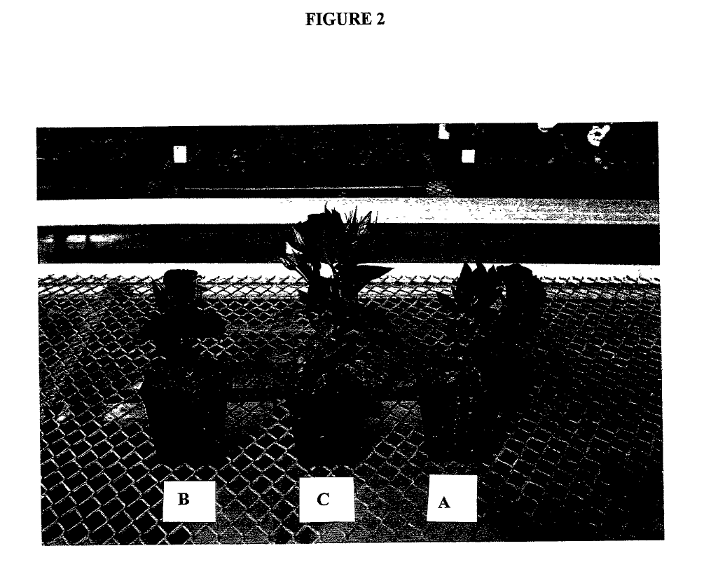

FIG. 2 is a photograph comparing the RAD Eustoma hybrid 3087 (labeled as “A” in the photograph) with commercial hybrids “Blue Lisa” (labeled as “B” in the photograph) and “Florida Blue” (labeled as “C” in the photograph).

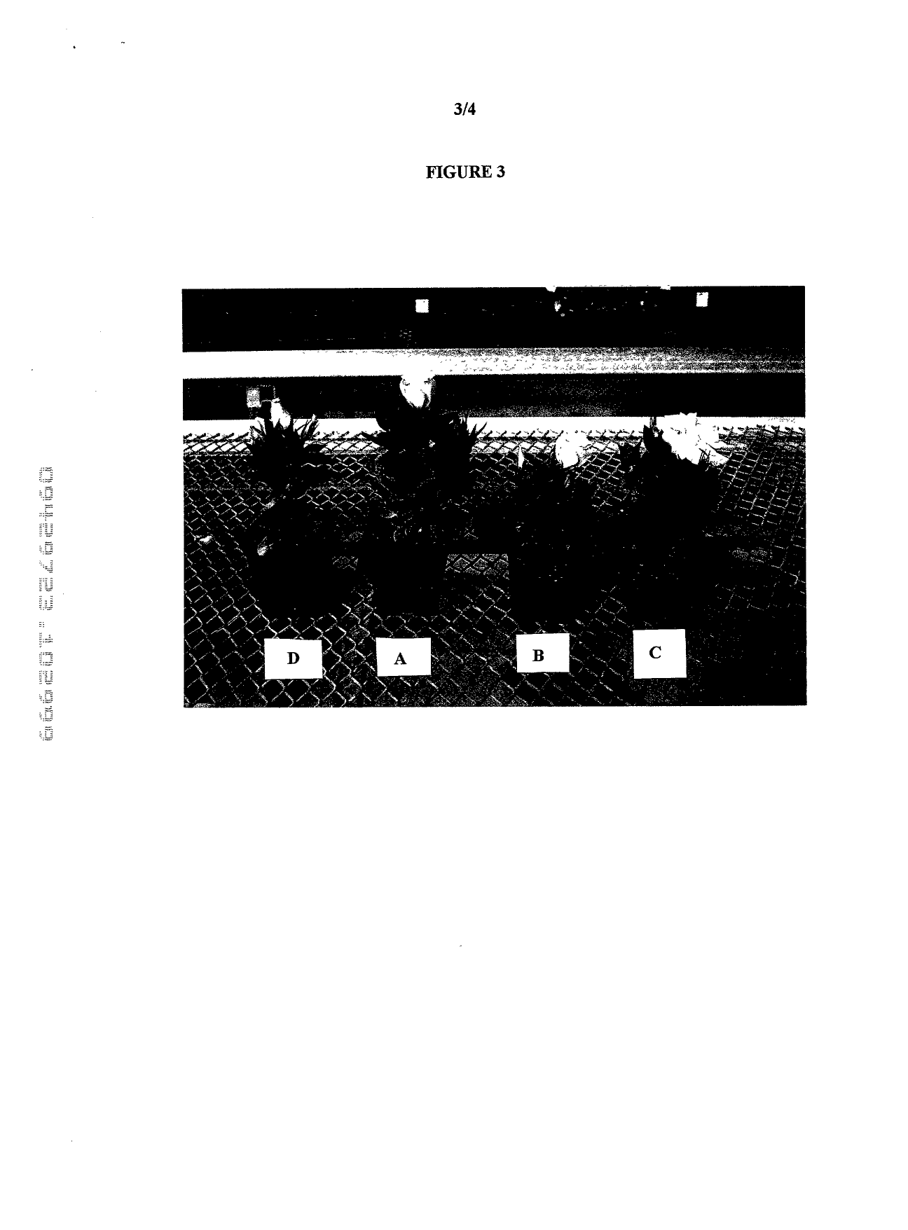

FIG. 3 is a photograph comparing the RAD Eustoma hybrids 3214 (labeled as “A” in the photograph), 3213 (labeled as “B” in the photograph) and 3211 (labeled as “C” in the photograph) with commercial hybrid “White Lisa” (labeled as “D” in the photograph).

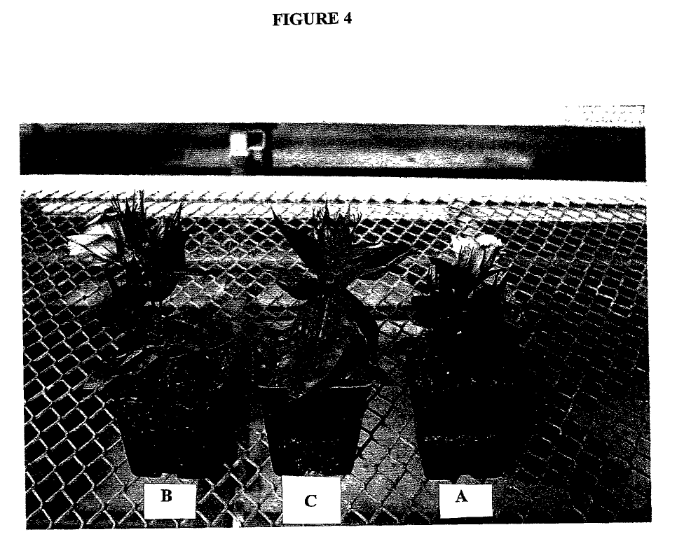

FIG. 4 is a photograph comparing the RAD Eustoma hybrid 3215 (labeled as “A” in the photograph) with commercial hybrids “Pink Lisa” (labeled as “B” in the photograph) and “Florida Pink” (labeled as “C” in the photograph).

### EXAMPLE 5

### Demonstration of Reduced Apical Dominance Inheritance

Hybrids were generated from crosses having reduced apical dominance lines used as either the male or female. Plants were grown using standard greenhouse conditions and evaluated for the RAD trait after four (4) months. Evidence of RAD inheritance is provided in Table 2, below. Chi square analysis yields a value of 0.89 demonstrating a fit for a 3:1 segregation ratio.

TABLE 2

| Hybrid | RAD Source | Total Plants | RAD Plants | % RAD |
|---|---|---|---|---|
| 3109-1 | 3003-1-1-2-1 | 188 | 54 | 29 |
| 3109-2 | 3003-1-1-2-1 | 200 | 45 | 23 |
| 3128-1 | 1389-1-1-14-2 | 341 | 72 | 21 |

Deposit Information

Eustoma seeds of hybrid 3087 have been placed on deposit with the American Type Culture Collection (ATCC), 10801 University Blvd., Manassus, Va., 20110-2209 under Deposit Accession Number 203392 on Oct. 30, 1998. This deposit was made in compliance with the Budapest Treaty requirements that the duration of the deposit should be for thirty (30) years from the date of deposit or for five (5) years after the last request for the deposit at the depository or for the enforceable life of a U.S. Patent that matures from this application, whichever is longer. These Eustoma seeds will be replenished should it become non-viable at the depository.

The present invention is illustrated by way of the foregoing description and examples. The foregoing description is intended as a non-limiting illustration, since many variations will become apparent to those skilled in the art in view thereof It is intended that all such variations within the scope and spirit of the appended claims be embraced thereby.

Changes can be made to the composition, operation and arrangement of the method of the present invention described herein without departing from the concept and scope of the invention as defined in the following claims.

What is claimed is:

1. A Eustoma plant having reduced apical dominance.

2. The Eustoma plant of claim 1 which contains an allele for reduced apical dominance.

3. A Eustoma plant having reduced apical dominance, wherein the plant has a pedigree which includes the plant 752 or derivatives thereof.

4. Seed of the plant of claims 1, 2 or 3.

5. Pollen of the plant of claims 1, 2 or 3.

6. An ovule of the plant of claims 1, 2 or 3.

7. A tissue culture comprising regenerable cells of the plant of claims 1 or 3.

8. A cutting of the plant of claims 1, 2 or 3.

9. Eustoma seed containing an allele for reduced apical dominance.

10. Eustoma seed containing an allele for reduced apical dominance, wherein the seed has a pedigree which includes the plant 752 or derivatives thereof.

11. A Eustoma plant produced by growing the seed of claims 9 or 10.

12. A reduced apical dominance allele which when expressed in a Eustoma plant produces a reduced apical dominant phenotype in said plant.

13. A method for producing F₁ hybrid Eustoma seed, the method comprising the steps of crossing a first parent Eustoma plant with a second parent Eustoma plant and harvesting the resultant F₁ hybrid Eustoma seed, wherein the first and second parent Eustoma plant is the Eustoma plant of claims 1, 2 or 3.

14. A first generation (F₁) hybrid Eustoma plant produced by growing the hybrid Eustoma seed of claim 13.

15. A process for identifying a Eustoma plant, the genome of which contains an allele for reduced apical dominance, the process comprising the steps of:
crossing a Eustoma plant to be tested with a Eustoma plant the genome of which contains an allele for reduced apical dominance;
recovering the resulting F₁ hybrid Eustoma seed;
planting the F₁ hybrid Eustoma seed and regenerating into plants; and
selecting Eustoma plants the genome of which contains an allele for reduced apical dominance and which exhibit a reduced apical dominance phenotype.

16. A process of obtaining hybrid Eustoma seed, the genome of which contains an allele for reduced apical dominance, the process comprising the steps of:
crossing a Eustoma plant obtained by the process of claim 15 with a Eustoma plant the genome of which contains an allele for reduced apical dominance; and
recovering the resulting hybrid Eustoma seed, the genome of which contains an allele for reduced apical dominance.

17. A process for obtaining a hybrid Eustoma plant, the genome of which contains an allele for reduced apical dominance, the process comprising the steps of:
crossing a Eustoma plant obtained by the process of claim 15 with a Eustoma plant the genome of which contains an allele for reduced apical dominance;
recovering the resulting hybrid Eustoma seed; and
planting the hybrid Eustoma seed and regenerating into plants, the genome of which contains the allele for reduced apical dominance.

18. Viable Eustoma seeds and plants and succeeding generations thereof grown from seeds deposited under ATCC Accession number 203392 and Eustoma seeds and plants to which the allele for reduced apical dominance is transferred from the deposited seeds in succeeding generations thereof.
